(19) United States
(12) Patent Application Publication
(10) Pub. No.: US 2005/0169877 A1
(43) Pub. Date: Aug. 4, 2005

(54) Process for preparing a composition using pressurized fluid, composition prepared, and uses thereof

(75) Inventors: Jean Francois Grollier, Paris (FR); Claude Dubief, Le Chesnay (FR); Roland De La Mettrie, Le Vesinet (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/040,002

(22) Filed: Jan. 24, 2005

Related U.S. Application Data
(60) Provisional application No. 60/545,186, filed on Feb. 18, 2004.

(30) Foreign Application Priority Data
Jan. 29, 2004 (FR) 0400848

(51) Int. Cl. A61K 7/06; A61K 7/075; A61K 7/08

(52) U.S. Cl. 424/70.31

(57) ABSTRACT

The present invention relates to a process for preparing a composition useful, for example, for the treatment of keratin materials, in particular the skin and human keratin fibres such as the hair, to the composition prepared, and also to a process for treating keratin materials using this composition.

### REFERENCE TO PRIOR APPLICATIONS

This application claims priority to U.S. provisional application 60/545,186 filed Feb. 18, 2004, and to French patent application 0400848 filed Jan. 29, 2004, both incorporated herein by reference.

### FIELD OF THE INVENTION

The present invention relates to a process for preparing a composition useful, for example, for the treatment of keratin materials, in particular the skin and human keratin fibres such as the hair, to the composition prepared, and also to a process for treating keratin materials using this composition.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

In cosmetics, it is always sought to improve the cosmetic properties of keratin materials, for example sensitized hair, i.e. hair that has been damaged or embrittled by the chemical action of atmospheric agents such as free radicals, light and pollution, and/or hair treatments such as permanent-waving, dyeing or bleaching. Cosmetic treatment compositions comprising conditioning agents such as esters, intended to repair or limit the harmful or adverse effects produced by the various treatments or attacking factors to which keratin materials are more or less repeatedly subjected, may thus be applied to these materials. These conditioning agents may also improve the cosmetic properties of natural hair.

Conditioning agents for keratin materials, such as esters, give wet or dry hair, in particular a good aptitude to disentangling, smoothness, softness and sheen.

These conditioning agents also give the skin cosmetic properties such as good moisturization and good nourishment.

However, cosmetic treatment compositions containing such conditioning agents are generally aqueous compositions in which the agents must be dissolved. The lack of solubility of these compounds reduces the conditioning power of these compositions. In addition, this solubility criterion reduces the number of esters that can be used for the cosmetic treatment of keratin materials. This is particularly the case for compounds with a high melting point.

### DETAILED DESCRIPTION OF THE INVENTION

It has now been discovered, surprisingly, that by using a novel preparation process, compositions that are useful, for example, as a cosmetic composition for treating keratin materials, more or less concentrated in conditioning agent(s), are obtained in a very short time, for example in less than 2 minutes, according to need, especially without preserving agent, enabling the solubility problems outlined above to be overcome.

This process can be performed simply and is appropriate to the needs of the consumer. A fluid, the temperature of which is preferably greater than or equal to 30° C., is passed, under pressure, for a very short time, for example less than 1 minute, through at least one conditioning agent in solid or pasty form, preferably solid and even more preferably pulverulent.

It also enables the use in anhydrous form of conditioning agents that are unstable in aqueous compositions either because they react with water or because they react in aqueous solution with compounds that do not react with them in an anhydrous composition. This is in particular the case of esters.

The compositions prepared according to this process may have limited stability on storage, which is not a drawback in this case since the process leads to a ready-to-use composition intended to be used quickly after its preparation, for example within five minutes of preparation, especially after cooling to a temperature which is acceptable for keratin materials, preferably below 60° C., better still below 50° C. The composition may also be used up to one week or more after its preparation, depending on the rate of degradation of the conditioning agent used.

Given the very short preparation time, the invention compositions may be prepared “on demand” by mixing different active compounds according to the desired (cosmetic) properties.

According to another embodiment, it is not necessary to determine beforehand the concentrations of said agents in solution, which limits the measuring errors by the user because the conditioning agents may be packaged in a ready-to-use device.

In addition, the process according to the invention makes it possible to avoid the use of multi-compartment bottles, which makes the process particularly economical and safer for the user.

The composition thus obtained may be used alone or as a mixture with another composition.

A further advantage of this preparation process is the production of compositions that have better cosmetic properties. In particular, keratin fibres treated with a composition obtained via the process according to the invention have improved conditioning properties, especially in terms of disentangling, smoothness, sheen and soft feel, and the skin treated in this way has improved softness, an improved smooth character and improved moisturization.

One subject of the invention is thus a process for preparing a composition preferably useful for treating keratin materials, comprising percolating a fluid at a pressure of at least 3 bar through at least one ester in solid or pasty form.

Another subject of the invention is a composition obtained via the process according to the invention.

A subject of the invention is also the use of the composition obtained according to the process of the invention, for the cosmetic treatment of keratin materials, and especially for conditioning the hair and the skin.

Another subject of the invention is a packaging device for performing the preparation process of the present invention.

Other subjects, characteristics, aspects and advantages of the invention will emerge even more clearly on reading the further, nonlimiting, description and the examples that follow.

According to the invention, the process for preparing a composition comprises a step of percolating fluid, at a temperature preferably of greater than or equal to 30° C., better still ranging from 30° C. to 150° C., and even more preferably from 40° C. to 120° C., at a pressure of at least 3 bar (3×105 Pa), through at least one ester in solid or pasty form.

Percolation is a movement of fluid through a porous medium, allowing the passage of the fluid due to the action or effect of pressure.

The fluid used in the process according to the invention preferably comprises at least steam. It may be steam optionally accompanied by liquid water, or a mixture of steam optionally accompanied by liquid water, and of one or more preferably cosmetically acceptable liquid and/or gaseous solvents. Preferably, the fluid is steam possibly accompanied by liquid water.

Examples of organic solvents that may be used include C1-C4 lower alcohols, such as ethanol and isopropanol; polyols and polyol ethers, for instance 2-butoxyethanol, propylene glycol, propylene glycol monomethyl ether, diethylene glycol monomethyl ether and diethylene glycol monoethyl ether, and also aromatic alcohols, for instance benzyl alcohol or phenoxyethanol, and mixtures thereof.

The ester is in solid or pasty form, preferably in solid form and even more preferably pulverulent.

For the purposes of the present invention, the term “pasty form” means a consistency intermediate between a solid phase and a liquid phase. The viscosity of this pasty phase is preferably greater than 0.1 Pa·s and even more preferably greater than 1 Pa·s, at 25° C. with a shear rate of 10 s−1.

The term “keratin materials” means the skin, the lips, and/or the integuments such as the nails and keratin fibres, for example the eyelashes, the eyebrows and the hair.

The process of the present invention may be performed using a standard device for generating a pressurized fluid at a temperature preferably of greater than or equal to 30° C., better still ranging from 30° C. to 150° C. and even more preferably from 40° C. to 120° C. Such a device comprises a pressure-resistant chamber equipped with a thermal block, and also a circuit for conveying the fluid to the conditioning agent used in the invention.

According to another embodiment, the device additionally comprises a reservoir of liquid(s) and also a pump for conveying the liquid(s) to the chamber.

The liquid contained in the reservoir is preferably either water, or a mixture of water and of one or more cosmetically acceptable solvents. Preferably, the liquid is water. A device that is particularly useful for performing the process of the present invention is a coffee machine of the “espresso” type. Such machines are well known in the art. For example, these machines are described in patents AT 168 405, U.S. Pat. No. 2,688,911, DE 324 33 870 and IT 1 265 636.

According to one particular embodiment of the invention, percolation is performed with a fluid at a temperature of greater than or equal to 30° C., preferably ranging from 30° C. to 150° C., and even more preferably from 40° C. to 120° C., under a pressure ranging from 3 to 30 bar (3×105 to 3×106 Pa), preferably of at least 4 bar (4×105 Pa), more preferably greater than or equal to 10 bar (106 Pa), and most particularly ranging from 10 to 30 bar (106 to 3×106 Pa).

A composition comprising at least one ester in solid or pasty form, may be used directly in the device for generating the pressurized fluid in a container intended for this use. It may also be packaged in a particular packaging device comprising a closed housing delimited by at least one wall that is at least partially permeable to fluid under a pressure of at least 3 bar. Such devices are described, for example, in patent applications WO 00/56629, EP 512 470, U.S. Pat. No. 5,897,899 or WO 99/03573. These packaging devices are generally protected against the air, humidity and/or light.

According to one particular embodiment, the housing is delimited by two sealed sheets. According to another embodiment, the housing is delimited by a tray closed with a lid.

These devices may be manufactured from woven or nonwoven, plastic or plant materials, for example cellulose, metal such as aluminium, or composite materials. Such devices are described, for example, in patent applications WO 00/56629, EP 512 470, U.S. Pat. No. 5,897,899 or WO 99/03573.

The esters that may be used in the present invention may be monomeric or polymeric. They are preferably non-silicone and non-cationic.

Examples of these esters that may especially be mentioned include:
esters of linear or branched C7-C30 monocarboxylic acids and of linear or branched C8-C30 monoalcohols, for instance cetyl palmitate, myristyl myristate, stearyl heptanoate, isostearyl behenate, cetyl isooctanoate, 2-decylmyristyl 2-decylmyristate and behenyl behenate;
esters of linear or branched C7-C30 unsaturated monocarboxylic acids or C6-C30 aromatic monocarboxylic acids and of linear or branched C8-C30 monoalcohols, for instance stearyl benzoate, cetyl ricinoleate, 2-octyldodecyl erucate and behenyl benzoate;
mono- and diesters of linear or branched C10-C30 monocarboxylic acids and of ethylene glycol, for instance ethylene glycol monostearate, ethylene glycol distearate and ethylene glycol dimyristate;
mono-, di- or triesters of linear or branched, saturated or unsaturated C10-C30 monocarboxylic acids and of a C3 polyol, for instance glyceryl mono-, di- and tristearate, glyceryl mono-, di- and tribehenate, propylene glycol monomyristate, glyceryl palmitostearate, glyceryl monooleate, glyceryl tristearobehenate, glyceryl triundecanoate, shea butter, Japan wax, illipe butter and cocoa butter;
mono- or polyesters of linear or branched, saturated or unsaturated C7-C30 monocarboxylic acids or C7-C30 aromatic monocarboxylic acids and of C4-C50 non-polyoxyalkylenated polyols, and in particular of sugars. Examples that may be mentioned include sucrose benzoate, pentaerythrityl tetraisostearate, pentaerythrityl tetrabehenate, pentaerythrityl tetralaurate, sorbitan palmitate, O-octanoyl-6′-D-maltose, sorbitan monostearate, sucrose laurate, neopentyl glycol dibenzoate, pentaerythrityl tetrabenzoate, esters of coconut fatty acids and of sorbitol, ditrimethylolpropane tetrastearate, dipentaerythrityl hexastearate, methylglucose mono- and distearate, sucrose dilaurate, dextrin palmitate, sucrose mono- and dipalmitostearate, methylglucose sesquistearate and hexylglucoside monococoate;
mono-, di- or triesters of linear or branched, saturated or unsaturated, aromatic or non-aromatic, hydroxylated or non-hydroxylated C4-C30 dicarboxylic acid or tricarboxylic acids and of linear or branched C7-C50 monools or polyols. Examples that may be mentioned include dibehenyl fumarate, the citric ester of tallow monoglycerides, dicocoyl-distearyl-pentaerythrityl citrate, dimyristyl tartrate, dihexyl, dioctyl, dilauryl or dicetyl mucate, dilauryl citrate and trilauryl citrate;
mono- or polyesters of polyoxyalkylenated or polyglycerolated acids and/or alcohols, the non-oxyalkylenated or non-glycerolated portion of the acids being of C10-C30, such as jojoba oil containing 80 mol of ethylene oxide (or EO) and jojoba wax containing 120 mol of EO, myristyl myristate containing 3 mol of EO, triethylene glycol monostearate and distearate, polyethylene glycol monostearates containing 8, 25, 30 or 100 mol of EO, polyethylene glycol distearates containing 8, 150 and 250 mol of EO, polyethylene glycol dibehenate containing 150 mol of EO, polyethylene glycol diisostearate containing 90 mol of EO, beeswaxes oxyethylenated with 6, 8 or 12 mol of EO, sorbitan monostearate oxyalkylenated with 4 mol of EO, sorbitan triisostearate oxyethylenated with 200 mol of EO, sorbitan hexastearate oxyethylenated with 6 mol of EO, methylglucoside dioleate oxyethylenated with 120 mol of EO, shea butter oxyethylenated with 75 mol of EO, castor oil oxyethylenated with 25 mol of EO, or palmitostearate polyglycerolated with 6 mol of glycerol;
hydrogenated oils or hydrogenated waxes, for instance hydrogenated rapeseed oil, macadamia oil, castor oil, cottonseed oil, soybean oil, jojoba oil, palm oil, rice bran oil or sunflower oil, and hydrogenated sunflower wax;
salts of acids resulting from the esterification of linear or branched, saturated or unsaturated C7-C30 monocarboxylic acids and of hydroxyl acids. Among these, mention may be made of sodium lauroyllactylate and sodium behenoyllactylate; polymers for which at least one of the monomers is an ester. These polymers may be homopolymers or copolymers. They may be of nonionic, anionic, cationic or amphoteric nature and may in particular be obtained by free-radical polymerization.

Among the homopolymers that may be particularly mentioned are poly(methyl methacrylates), poly(vinyl acetates), polycaprolactones, poly(stearyl acrylates) and poly(ethyl acrylates).

Among the copolymers that may be particularly mentioned are copolymers of vinyl acetate and of ethylene, vinyl acetate/vinyl versatate/butyl acrylate copolymers, acrylic acid/stearyl methacrylate copolymers, N-octylacrylamide/methyl methacrylate/hydroxypropyl methacrylate/acrylic acid/tert-butylaminoethyl methacrylate copolymers, lactic acid/vinylpyrrolidone/lauryl methacrylate copolymers; and
polymers resulting from the polycondensation of at least one polyacid and of at least one polyol. Among these polymers, mention may be made of poly(neopentyl glycol terephthalate), diglycol/cyclohexanedimethanol/isophthalates/sulfoisophthalates polymers, polyesters of polyglycerol and of eicosanedioic acid, polyesters of trimethylolpropane and of dimethylolpropionic acid, polymers of polyethylene glycol and of adipic acid, and polymers of sebacic acid and of butylene glycol.

The esters that are particularly preferred in the present invention are chosen from esters of linear or branched C7-C30 monocarboxylic acids and of linear or branched C8-C30 monoalcohols; esters of linear or branched C7-C30 unsaturated monocarboxylic acids or C6-C30 aromatic monocarboxylic acids and of linear or branched C8-C30 monoalcohols; mono- and diesters of linear or branched C10-C30 monocarboxylic acids and of ethylene glycol; mono-, di- or triesters of linear or branched, saturated or unsaturated C10-C30 monocarboxylic acids and of a C3 polyol; mono- or polyesters of linear or branched, saturated or unsaturated, or aromatic C7-C30 monocarboxylic acids and of non-polyoxyalkylenated C4-C50 polyols; and polymers for which at least one of the monomers is an ester, as described above.

The esters may be used as a mixture, and may be used as a mixture with one or more solid or pasty, and preferably pulverulent, adjuvants. The adjuvants may be chosen for example from clays, salts, anionic, nonionic, cationic or zwitterionic surfactants, natural or synthetic thickeners, optionally modified starch, glass beads, silica, Nylon, alumina, titanium dioxide, zeolites, polymethyl methacrylate (PMMA), chitosan, maltodextrin, cyclodextrin, mono- or disaccharides, for instance glucose, sucrose, sorbitol or fructose, zinc oxide, zirconium oxide, silica beads, talc, polyaspartic acid, borosilicates, especially calcium borosilicate, polyethylene, cotton, polytetrafluoroethylene (PTFE), cellulose and its derivatives, superabsorbent compounds, magnesium carbonate, calcium carbonate, corn seeds, polyacrylamide, porous hydroxyapatite, silk, collagen, sawdust, wrack powder, meals or extracts of wheat, rice, pea, lupin, soybean or barley, crosslinked polyvinylpyrrolidone, calcium alginate, active charcoal, and poly(vinylidene chloride/acrylonitrile) particles, especially those sold under the general name “Expancel®” by the company Akzo Nobel under the particular reference “Expancel® WE” or “Expancel DE”, and mixtures thereof.

When one or more adjuvants are present, the ester(s) used in the invention is (are) preferably present in an amount ranging from 0.5% to 99% by weight, better still from 1% to 80% by weight and even more preferably from 2% to 60% by weight relative to the total weight of ester(s) and adjuvant(s) in solid or pasty form.

When plants or plant extracts are used in the process of the present invention, they may be pretreated before the percolation step. The pretreatment can be drying, roasting, cryogrinding or freeze-drying.

The (cosmetic) composition for treating keratin materials obtained according to the process of the invention preferably contains, besides the ester(s) and the component(s) of the fluid, i.e. water and/or cosmetically acceptable solvent(s), optionally all or some of the adjuvant(s) present in the solid or pasty mixture.

The invention also relates to a composition that may be obtained via the process according to the invention, a particularly preferred composition not comprising any preserving agents.

Using the preparation process of the invention, a composition for treating keratin materials is obtained, which may be applied directly to keratin materials, or which may be mixed with a cosmetically acceptable medium, or alternatively at least one additive conventionally used in cosmetics may be added thereto by an operator. At least two compositions obtained via the process of the invention may also be mixed together. The cosmetic composition for treating keratin materials optionally resulting from the mixture(s) and/or addition(s) indicated above will be referred to hereinbelow as the final (cosmetic treatment) composition or final composition.

One particular embodiment of the invention is applying the composition obtained by means of a device not requiring any human intervention, and optionally equipped with a cooling means.

The amount of the ester(s) present in the final (cosmetic treatment) composition is generally between 0.001% and 50% by weight approximately, preferably between 0.005% and 30% by weight, and even more preferably between 0.01% and 20% by weight, relative to the total weight of the final (cosmetic treatment) composition.

When the composition obtained via the process of the present invention is mixed with a cosmetically acceptable medium, such a medium generally preferably comprises water or a mixture of water and of at least one organic solvent to dissolve the compounds that would not be sufficiently soluble in water.

The term “cosmetically acceptable” means a medium which is compatible with keratin materials and especially the skin, the lips and/or the integuments, and which additionally has an appearance, a feel, a smell or optionally a flavour which are pleasant for the user.

Examples of organic solvents that may be particularly mentioned include C1-C4 lower alcohols, such as ethanol and isopropanol; polyols and polyol ethers, for instance 2-butoxyethanol, propylene glycol, propylene glycol monomethyl ether and diethylene glycol monomethyl ether and diethylene glycol monoethyl ether, and also aromatic alcohols, for instance benzyl alcohol or phenoxyethanol, and mixtures thereof.

The solvents are preferably present in proportions preferably of between 1% and 40% by weight approximately and even more preferably between 5% and 30% by weight approximately relative to the total weight of the final (cosmetic treatment) composition.

At least one additive conventionally used in cosmetics may also be added to the cosmetic treatment compositions obtained according to the process of the present invention. Examples of such additives that may be mentioned include anionic, cationic, nonionic, amphoteric or zwitterionic surfactants, or mixtures thereof; anionic, cationic, nonionic, amphoteric or zwitterionic polymers, or mixtures thereof, mineral or organic thickeners, and in particular anionic, cationic, nonionic and amphoteric polymeric associative thickeners; antioxidants; penetrating agents; sequestering agents; fragrances; buffers; dispersants; conditioning agents other than those described above, for instance silicone oils; film-forming agents; preserving agents and opacifiers.

The above additives are generally present in an amount for each of them of between 0.01% and 20% by weight relative to the weight of the final composition.

Needless to say, a person skilled in the art will take care to select this or these optional additional compounds such that the advantageous properties intrinsically associated with the cosmetic treatment composition in accordance with the invention are not, or are not substantially, adversely affected by the addition(s) envisaged.

The pH of the final (cosmetic treatment) composition is generally preferably between 3 and 12 and preferably between 5 and 11. It may be adjusted to the desired value using acidifying or basifying agents usually used in cosmetics, or alternatively using standard buffer systems.

Among the acidifying agents that may be mentioned, for example, are mineral or organic acids such as hydrochloric acid, orthophosphoric acid, sulfuric acid, carboxylic acids such as acetic acid, tartaric acid, citric acid and lactic acid, and sulfonic acids.

Among the basifying agents that may be mentioned, for example, are ammonia, alkaline carbonates, alkanolamines such as mono-, di- and triethanolamine and derivatives thereof, sodium hydroxide, potassium hydroxide and the compounds of the following formula: 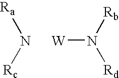
in which W is a propylene residue that is optionally substituted with a hydroxyl group or a C1-C4 alkyl radical; Ra, Rb, Rc and Rd, which may be identical or different, represent a hydrogen atom, a C1-C4 alkyl radical or a C1-C4 hydroxyalkyl radical.

The final (cosmetic treatment) composition may be in any of various forms, such as in the form of liquids, creams or gels, or in any other form that is suitable for treating keratin materials, and especially the skin or the hair, and more particularly the hair.

The final (cosmetic treatment) composition may be used, for example, as a shampoo, a rinse-out or leave-in conditioner, a deep-down care mask, a shower gel, or a lotion or cream for treating keratin materials.

The present invention also relates to a cosmetic process for treating keratin materials, comprising the preparation of a cosmetic treatment composition according to the process as defined above, and its application to the keratin materials, for example by means of an operator or by means of a device not requiring any human intervention. The application time may range for example between 15 seconds and one hour.

Before application, the cosmetic treatment composition obtained according to the process of the invention may be mixed with a cosmetically acceptable medium and/or with one or more additives conventionally used in cosmetics, as described above.

Another embodiment of the invention is preparing at least two cosmetic treatment compositions according to the process of the invention, mixing them together, and optionally adding a cosmetically acceptable medium and/or one or more additives conventionally used in cosmetics, as described above, and then in applying the final composition obtained to keratin materials.

The examples below illustrate the present invention, but do not limit it.

### EXAMPLE 1

The solid ingredients below are mixed together in the proportions indicated as weight percentages relative to the total weight of solid mixture:

| | |
|---|---|
| Myristyl myristate | 30% |
| Dipalmitoylethyl hydroxyethyl | 30% |
| methylammonium methosulfate/cetearyl alcohol sold under the | |
| trade name Dehyquart ® F30 by the company Cognis | |
| Sucrose palmitostearate | 40% |

5 g of this mixture are placed in a commercial espresso machine. Steam is then passed through until a composition (A) having a final volume of 50 ml is obtained.

A cosmetic treatment composition ready to be applied to the hair is thus obtained.

Improved smoothness and softness of the hair is thus obtained.

Two parts by weight of composition (A) may be added to one part by weight of an aqueous composition (B) containing 1% by weight of hydroxyethylcellulose, to facilitate the application.

### EXAMPLE 2

The solid ingredients below are mixed together in the proportions indicated as weight percentages relative to the total weight of solid mixture:

| | |
|---|---|
| Ethyleneglycol distearate | 10% |
| Carboxymethylstarch (potato flour) in the form | 20% |
| of weakly crosslinked sodium salt, sold under the trade name | |
| Primojel by the company Avebe | |
| Sodium laurylsulfate in powder form sold | 70% |
| under the trade name Texapon ® Z 95 P by the company Cognis | |

5 g of this mixture are placed in a commercial espresso machine. Steam is then passed through until a composition (A) having a final volume of 50 ml is obtained.

A cosmetic treatment composition ready to be applied to the hair is thus obtained.

Improved smoothness and softness of the hair is thus obtained.

Two parts by weight of composition (A) may be added to one part by weight of an aqueous composition (B) containing 1% by weight of hydroxyethylcellulose, to facilitate the application.

The above written description of the invention provides a manner and process of making and using it such that any person skilled in this art is enabled to make and use the same, this enablement being provided in particular for the subject matter of the appended claims, which make up a part of the original description and including a process for preparing a cosmetic composition for treating keratin materials, wherein it comprises a step of percolating fluid comprising at least steam, at a pressure of at least 3 bar through at least one ester in solid or pasty form.

As used herein, the phrases “selected from the group consisting of,” “chosen from,” “selected from,” and the like include mixtures of the specified materials.

Where compounds are described as, e.g., “cellulose and its derivatives” an alternate is “cellulose and cellulose compounds” where cellulose derivatives and compounds share a common core/structure with cellulose. One of ordinary skill in the art knows how to identify derivatives and compounds of compound X based on the structure of X and the similarity in structure of the derivatives and compounds.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A process for preparing a composition, comprising percolating a fluid comprising steam at a pressure of at least 3 bar through at least one ester in solid or pasty form.

2. A process according to claim 1, wherein the fluid further comprises at least one selected from liquid water, one or more cosmetically acceptable liquid and/or gaseous solvents, and mixtures thereof.

3. A process according to claim 1, wherein the ester is selected from esters of linear or branched C7-C30 monocarboxylic acids and of linear or branched C8-C30 monoalcohols; esters of linear or branched C7-C30 unsaturated monocarboxylic acids or aromatic C6-C30 monocarboxylic acids and of linear or branched C8-C30 monoalcohols; mono- and diesters of linear or branched C10-C30 monocarboxylic acids and of ethylene glycol; mono-, di- or triesters of linear or branched, saturated or unsaturated C10-C30 monocarboxylic acids and of a C3 polyol; mono- or polyesters of linear or branched, saturated or unsaturated, or aromatic C7-C30 monocarboxylic acids and of non-polyoxyalkylenated C4-C50 polyols; mono-, di- or triesters of linear or branched, saturated or unsaturated, aromatic or non-aromatic, hydroxylated or non-hydroxylated C4-C30 dicarboxylic or tricarboxylic acids and of linear or branched C7-C50 monools or polyols; mono- or polyesters of polyoxyalkylenated or polyglycerolated acids and/or alcohols, the non-oxyalkylenated or non-glycerolated portion of the acids being of C10-C30; hydrogenated oils or hydrogenated waxes; salts of acids resulting from the esterification of linear or branched, saturated or unsaturated C7-C30 monocarboxylic acids and of hydroxyl acids; polymers for which at least one of the monomers is an ester; polymers resulting from the polycondensation of at least one polyacid and of at least one polyol, and mixtures thereof.

4. A process according to claim 3, wherein the ester is selected from cetyl palmitate, myristyl myristate, stearyl heptanoate, isostearyl behenate, cetyl isooctanoate, 2-decylmyristyl 2-decylmyristate and behenyl behenate; stearyl benzoate, cetyl ricinoleate, 2-octyldodecyl erucate and behenyl benzoate; ethylene glycol monostearate, ethylene glycol distearate and ethylene glycol dimyristate; glyceryl mono-, di- and tristearate, glyceryl mono-, di- and tribehenate, propylene glycol monomyristate, glyceryl palmitostearate, glyceryl monooleate, glyceryl tristearobehenate, glyceryl triundecanoate, shea butter, Japan wax, illipe butter and cocoa butter; sucrose benzoate, pentaerythrityl tetraisostearate, pentaerythrityl tetrabehenate, pentaerythrityl tetralaurate, sorbitan palmitate, O-octanoyl-6′-D-maltose, sorbitan monostearate, sucrose laurate, neopentyl glycol dibenzoate, pentaerythrityl tetrabenzoate, esters of coconut fatty acids and of sorbitol, ditrimethylolpropane tetrastearate, dipentaerythrityl hexastearate, methylglucose mono- and distearate, sucrose dilaurate, dextrin palmitate, sucrose mono- and dipalmitostearate, methylglucose sesquistearate and hexylglucoside monococoate; dibehenyl fumarate, the citric ester of tallow monoglycerides, dicocoyl-distearylpentaerythrityl citrate, dimyristyl tartrate, dihexyl, dioctyl, dilauryl or dicetyl mucate, dilauryl citrate and trilauryl citrate; jojoba oil containing 80 mol of ethylene oxide (or EO) and jojoba wax containing 120 mol of EO, myristyl myristate containing 3 mol of EO, triethylene glycol monostearate and distearate, polyethylene glycol monostearates containing 8, 25, 30 or 100 mol of EO, polyethylene glycol distearates containing 8, 150 and 250 mol of EO, polyethylene glycol dibehenate containing 150 mol of EO, polyethylene glycol diisostearate containing 90 mol of EO, beeswaxes oxyethylenated with 6, 8 or 12 mol of EO, sorbitan monostearate oxyalkylenated with 4 mol of EO, sorbitan triisostearate oxyethylenated with 200 mol of EO, sorbitan hexastearate oxyethylenated with 6 mol of EO, methylglucoside dioleate oxyethylenated with 120 mol of EO, shea butter oxyethylenated with 75 mol of EO, castor oil oxyethylenated with 25 mol of EO, or palmitostearate polyglycerolated with 6 mol of glycerol; hydrogenated rapeseed oil, macadamia oil, castor oil, cottonseed oil, soybean oil, jojoba oil, palm oil, rice bran oil or sunflower oil, and hydrogenated sunflower wax; sodium lauroyllactylate and sodium behenoyllactylate; poly(methyl methacrylates), poly(vinyl acetates), polycaprolactones, poly(stearyl acrylates) and poly(ethyl acrylates), copolymers of vinyl acetate and of ethylene, vinyl acetate/vinyl versatate/butyl acrylate copolymers, acrylic acid/stearyl methacrylate copolymers, N-octylacrylamide/methyl methacrylate/hydroxypropyl methacrylate/acrylic acid/tert-butyl amino ethyl methacryl ate copolymers, lactic acid/vinylpyrrolidone/lauryl methacrylate copolymers; and poly(neopentyl glycol terephthalate), diglycol/cyclohexanedimethanol/isophthalates/sulfoisophthalates polymers, polyesters of polyglycerol and of eicosanedioic acid, polyesters of trimethylolpropane and of dimethylolpropionic acid, polymers of polyethylene glycol and of adipic acid, polymers of sebacic acid and of butylene glycol, and mixtures thereof.

5. A process according to claim 1, wherein the ester in solid or pasty form is present in a mixture with at least one adjuvant.

6. A process according to claim 5, wherein the adjuvant is selected from clays, salts, anionic, nonionic, cationic or zwitterionic surfactants, natural or synthetic thickeners, optionally modified starch, glass beads, silica, Nylon, alumina, titanium dioxide, zeolites, polymethyl methacrylate (PMMA), chitosan, maltodextrin, cyclodextrin, mono- or disaccharides, zinc oxide, zirconium oxide, silica beads, talc, polyaspartic acid, borosilicates, polyethylene, cotton, polytetrafluoroethylene (PTFE), cellulose and its derivatives, superabsorbent compounds, magnesium carbonate, calcium carbonate, corn seeds, polyacrylamide, porous hydroxyapatite, silk, collagen, sawdust, wrack powder, meals or extracts of wheat, rice, pea, lupin, soybean or barley, crosslinked polyvinylpyrrolidone, calcium alginate, active charcoal, poly(vinylidene chloride/acrylonitrile) particles, and mixtures thereof.

7. A process according to claim 5, wherein the ester(s) is (are) present in an amount of from 0.5% to 99% by weight relative to the total weight of ester(s) and adjuvant(s) in solid or pasty form.

8. A process according to claim 5, wherein the composition obtained comprises, in addition to the ester(s) and the component(s) of the fluid, all or some of the adjuvant(s) present in the mixture.

9. A process according to claim 1, wherein percolation is performed with a fluid at a pressure of 3 to 30 bar.

10. A process according to claim 9, wherein percolation is performed with a fluid at a pressure of at least 10 bar.

11. A composition prepared according to the process of claim 1.

12. The composition according to claim 11, which does not comprise preserving agent.

13. A process for treating keratin materials, comprising applying a composition prepared according to the process of claim 1 to keratin material.

14. The process according to claim 13, wherein the composition is applied to the keratin material by a device not requiring any human intervention.

15. The process according to claim 13, wherein, before application, the composition prepared according to the process of claim 1 is mixed with a cosmetically acceptable medium and/or with one or more additives.

16. The process according to claim 13, wherein, before application, the composition prepared according to the process of claim 1 is mixed with another composition prepared according to the same process and the mixture is applied to the keratin material.

17. A device comprising a closed housing delimited by at least one wall that is at least partially permeable to fluid at a pressure of at least 3 bar, the device containing therein at least one ester in solid or pasty form.

18. The device according to claim 17, in which the housing is delimited by two sealed sheets.

19. The device according to claim 17, in which the housing is delimited by a tray closed with a lid.
